(19) United States
(12) Patent Application Publication
(10) Pub. No.: US 2009/0306427 A1
(43) Pub. Date: Dec. 10, 2009

(54) Chemical Functionalization of Carbon Nanotubes

(76) Inventors: Yadienka Martinez-Rubi, Ottawa (CA); Jingwen Guan, Ottawa (CA); Benoit Simard, Orleans (CA)

Correspondence Address: SIM & MCBURNEY, 330 UNIVERSITY AVENUE, 6TH FLOOR, TORONTO, ON, M5G 1R7, CA

(21) Appl. No.: 12/224,438

(22) PCT Filed: Feb. 26, 2007
(86) PCT No.: PCT/CA2007/000297

§ 371 Date: Feb. 2, 2009

Related U.S. Application Data
(60) Provisional application No. 60/777,570, filed on Mar. 1, 2006.

(51) Int. Cl. C07C 55/02; C07C 15/24; C07C 2/56

(52) U.S. Cl. 562/590; 585/446; 585/709; 977/746

(57) ABSTRACT

The invention relates to a process for chemically functionalizing carbon nanotubes. The process comprises dispersing carbon nanotube salts in a solvent; and chemically functionalizing the carbon nanotube salts to provide chemically functionalized carbon nanotubes.

### FIELD OF THE INVENTION

The present invention relates to carbon nanotubes. In particular, the present invention relates to the chemical functionalization of carbon nanotubes.

### BACKGROUND OF THE INVENTION

There has been a great deal of interest in chemical functionalization of carbon nanotubes in order to facilitate manipulation, to enhance their solubility, and to make them more amenable to composite formation. Carbon nanotubes possess tremendous strength, an extreme aspect ratio, and are excellent thermal and electrical conductors. In view of these properties, chemically modified carbon nanotubes can be useful in many applications, for example, in polymer composite materials, molecular electronic applications and sensor devices. Because of their high crystallinity and high aromaticity, carbon nanotubes are substantially chemically inert and hence, difficult to be chemically functionalized for such applications. Conventionally, chemical functionalization of carbon nanotubes was possible under a very harsh oxidative environment, such as in highly concentrated boiling acids; through halogenation, particularly with fluorine gas; or through very limited nucleophilic and electrophilic reactions.

Most reaction procedures for chemical functionalization of carbon nanotubes, however, required long reaction times, ranging from several hours to several days. In addition, during such procedures, the carbon nanotubes were overly exposed to harsh media such that the carbon nanotubes were damaged and, very often, severely shortened. Moreover, the carbon nanotubes remained bundled together so that functionalization occurred only on the surface of the bundles, leaving the internal carbon nanotubes of the bundles unfunctionalized.

Functionalization with neutral carbon nanotubes can occur with oxidizing agents or thermally unstable, radical producing species, such as ozone, dimethylsulfoxide (DMSO), peroxides, azo and diazonium salts, and stable radicals such as NO (nitric oxide). Reactions of most of these species with neutral carbon nanotubes have been demonstrated in U.S. Patent Application Publication No. 2004/0223900 to Khabashesku et al.; U.S. Patent Application Publication No. 200510229334 to Huang et al.; and U.S. Patent Application Publication No. 2004/0071624 to Tour et al., the disclosures of which are incorporated herein by reference, but it requires several hours, even days, to achieve sufficient functionalization.

In J. Am. Chem. Soc., 127, 14867 (2005) to Tour et al., the disclosure of which is incorporated herein by reference, rapid chemical functionalization of single-walled carbon nanotubes has been shown. In particular, ionic liquids are used to debundle the carbon nanotubes and aryldiazonium salts are used to functionalize the carbon nanotubes. This process is limited, however, to diazonium salts and the ionic liquid.

Therefore, there is a need to develop a process for chemical functionalization of carbon nanotubes that obviates and mitigates at least some of the disadvantages of the prior art processes.

### SUMMARY OF THE INVENTION

In an aspect, there is provided a process for chemically functionalizing carbon nanotubes, the process comprising: dispersing carbon nanotube salt in a solvent; and chemically functionalizing the carbon nanotube salt to provide chemically functionalized carbon nanotubes.

In another aspect, dispersing the carbon nanotube salt in the solvent comprises chemically reducing carbon nanotubes to the carbon nanotube salt. The carbon nanotube salt comprises negatively charged carbon nanotubes.

In yet another aspect, chemically reducing the carbon nanotubes to the carbon nanotube salt comprises addition of a radical ion salt of formula A⁺B⁻ to the carbon nanotubes in the solvent, wherein A⁺ is a cation of an alkali metal and B⁻ is a radical anion of a polyaromatic compound.

In another aspect, the alkali metal is lithium, potassium, and/or sodium. In a further aspect, the polyaromatic compound is naphthalene and/or benzophenone. In still a further aspect, the solvent is a polar organic solvent.

In yet another aspect, the chemically functionalized carbon nanotubes comprise functional groups selected from —COOH, —PO₄⁻, —SO₃³¹ , —SO₃H, —SH, —NH₂, tertiary amines, quaternary amines, —CHO, —OH, alkyl, alkenyl, alkynyl, cycloalkyl, heterocyclyl, cycloalkenyl, alkoxy, alkanoyl, acyl, aryl, and/or heteroaryl groups.

In another aspect, chemically functionalizing the carbon nanotube salt comprises reacting oxidizing agents or thermally unstable, radical producing species with the carbon nanotube salt.

In yet another aspect, chemically functionalizing the carbon nanotube salt comprises reacting ozone, dimethylsulfoxide, peroxides, azo compounds, or diazonium compounds with the carbon nanotube salt in another aspect, the degree of functionalization is 1 functional group per 100 nanotube carbons. In a further aspect, the process is a single-pot process. In yet another aspect, the reaction time of functionalizing the carbon nanotube salt is about 30 minutes or less.

In a further aspect, the carbon nanotubes are selected from SWNTs, DWNTs and/or MWNTs. In another aspect, chemical functionalizing of the process occurs at a temperature that initiates chemical functionalization. In another aspect, the process occurs at about room temperature. In yet another aspect, the carbon nanotube salt is a chemically functionalized carbon nanotube salt.

In yet a further aspect, the chemically functionalized carbon nanotubes resulting from the process are converted to a chemically functionalized carbon nanotube salt, which now is the carbon nanotube salt when the process is repeated.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

Certain embodiments of the present invention will now be described more fully with reference to the accompanying drawings:

FIG. 1 is an embodiment showing the formation of a dispersion of a sodium salt of CNTs;

FIG. 2 is an embodiment showing the functionalization of a sodium salt of CNTs;

FIG. 3 is a Raman spectrum showing functionalization with dibenzoyl peroxide in an embodiment of the invention;

FIG. 4 is a Raman spectrum showing functionalization with lauroyl peroxide in an embodiment of the invention;

FIG. 5 is a Raman spectrum showing functionalization with lauroyl peroxide in the embodiment shown in FIG. 4, after reflux;

FIG. 6 is a Raman spectrum showing functionalization with glutaric. acid acyl peroxide in an embodiment of the invention;

FIG. 7 is infrared spectra of pristine SWNT, SWNT functionalized with glutaric (SWNT-GAP) and succinic (SWNT-SAP) acid acyl peroxide; and

FIG. 8 is a Raman spectrum showing functionalization with DMSO in an embodiment of the invention.

### DETAILED DESCRIPTION OF CERTAIN EMBODIMENTS

The following definitions are used herein and should be referred to for interpretation of the claims and the specification:

“CNT” means carbon nanotube; “SWNT” means single-walled nanotube; “DWNT” means double-walled nanotube; and “MWNT” means multi-walled nanotube.

The term “dispersing”, “dissolution” and the like refers to substantially debundling carbon nanotubes, ropes to substantially distribute homogeneously the carbon nanotubes in solvents.

The term “chemically functionalized carbon nanotubes” and the like refers to functional groups covalently bonded to the surface of CNTs.

The term “carbon nanotube” refers to a hollow article composed primarily of carbon atoms. Typically, single-walled carbon nanotubes are about 0.5 to 2 nm in diameter where the ratio of the length dimension to the narrow dimension (diameter), i.e., the aspect ratio, is at least 5. In general, the aspect ratio is between 10 and 2000. Carbon nanotubes are comprised primarily of carbon atoms; however, they may be doped with other compounds/elements, for example, and without being limited thereto, metals, boron, nitrogen and/or others. The carbon-based nanotubes of the invention can be multi-walled nanotubes (MWNTs), double-walled nanotube (DWNTs) or single-walled nanotubes (SWNTs). A MWNT, for example, includes several concentric nanotubes each having a different diameter. Thus, the smallest diameter tube is encapsulated by a larger diameter tube, which in turn, is encapsulated by another larger diameter nanotube. A DWNT includes two concentric nanotubes and a SWNT includes only one nanotube.

Carbon nanotubes may be produced by a variety of methods, and are commercially available, for example, from Carbon Nanotechnologies Inc. (Houston, Tex.) and Carbon Solutions Inc. (Riverside, Calif.). Methods of CNT synthesis include laser vaporization of graphite (A. Thess et al. Science 273, 483 (1996)), arc discharge (C. Joumet et al., Nature 388, 756 (1997)) and HiPCo (high pressure carbon monoxide) process (P. Nikolaev et al., Chem. Phys. Lett. 313, 91-97 (1999)). Chemical vapor deposition (CVD) can also be used in producing carbon nanotubes (J. Kong et al., Chem. Phys. Lett. 292, 567-574 (1998): J. Kong et al., Nature 395, 878-879 (1998); A. Cassell et al., J. Phys. Chem. 103, 6484-4492 (1999); and H. Dai et al., J. Phys. Chem. 103, 11246-11255 (1999)).

Additionally CNTs may be grown via catalytic processes both in solution and on solid substrates (Yan Li, et al., Chem. Mater. 13(3), 1008-1014 (2001); N. Franklin and H. Dai Adv. Mater. 12, 890 (2000); and A. Cassell et al., J. Am. Chem. Soc. 121, 7975-7976 (1999)). Most CNTs, as presently prepared, are in the form of entangled tubes. Individual tubes in the product differ in diameter, chirality, and number of walls. Moreover, long tubes show a strong tendency to aggregate into “ropes” held together by Van der Waals forces. These ropes are formed due to the large surface areas of nanotubes and can contain a few to hundreds of nanotubes in one rope.

The present invention is directed to a process for producing chemically functionalized CNTs. The process comprises dispersing CNTs and functionalizing the CNTs. In an embodiment, the process comprises dispersing CNT salt and functionalizing the CNT salt. In a specific embodiment, the process comprises chemically reducing the CNTs to negatively charged CNTs for dispersion and chemical functionalization.

In certain embodiments, the process and materials of the invention can reduce reaction times from days and hours to minutes, producing covalently functionalized CNTs at the SWNT level. Similarly, this can also be achieved with DWNTs and MWNTs.

The process of dispersing the CNT and chemical functionalization of carbon nanotubes can be achieved in a single-pot process; can provide covalently functionalized CNTs; can be efficient and take place within minutes; can be conducted at room temperature; and can control the degree and type of functionalization.

### Dispersion

The process of the invention comprises dispersing CNTs prior to functionalization. Dispersion can be effected by a process developed by Penicaud et al. and described in International Patent Application No. WO 2005/073127 and the J. Amer. Chem. Soc., 127, 8 (2005). each disclosure of which is incorporated by reference. By using alkali salts, this process negatively ionizes the CNTs to form a dispersion. The CNTs become reducing agents. Such a dispersion process is particularly applicable to SWNTs.

As described in International Patent Application No. WO 2005/073127, the dissolution of CNTs involves the reduction of CNTs, which leads to negatively charged nanotubes and positively charged counter-ions. In a typical embodiment, the positively charged counter-ions are cations of alkali metals, such as lithium, potassium, sodium and/or rubidium. The process includes the addition of a radical Ion salt of formula A⁺B⁻ to the CNTs in a polar organic solvent, wherein A⁺ is a cation of an alkali metal, such as lithium, potassium, sodium and/or rubidium, and B⁻ is a radical anion of a polyaromatic compound. The radical anion of the polyaromatic compound acts as an electron carrier to reduce the CNTs to negatively charged CNT salts. Any suitable polyaromatic compound can be used in this process that is capable of acting as an electron carrier to reduce the CNTs to negatively charged CNT salts. For example and without being limited thereto, the polyaromatic compound can be selected from naphthalene and/or benzophenone. Any suitable polar organic solvent(s) that can be used in this process involving electron transfer to reduce the CNTs to negatively charged CNT salts. For example and without being limited thereto, the solvent can be tetrahydrofuran (THF), ethers, 1,2-dimethoxyethane (DME), toluene, and/or pyridine.

A particular embodiment includes the synthesis of a lithium salt of CNTs. The reaction takes place in an inert atmosphere, for example, under argon. The CNT salts are obtained by reaction of a suspension of carbon nanotubes in THF in which is dissolved a lithium naphthalene salt, according to Petit et al., Chem. Phys. Lett., 305, 370 (1999) and Jouguelet et al., Chem. Phys. Lett., 318, 561 (2000). The lithium naphthalene salt was prepared by reaction of naphthalene with an excess of lithium in THF until a very dark color green forms. This salt-solution was then added to CNTs and stirred for a few hours. More specifically, about 320 mg of naphthalene and about 30 mg of lithium are combined in a flask and about 100 ml of THF is added thereto. The mixture is refluxed until the mixture forms a very dark green colour and left to reflux for a few hours. The lithium naphthalene salt solution is filtered to remove excess lithium. About 220 mg of CNTs are added to the lithium naphthalene salt filtrate and stirred for about 4 hours.

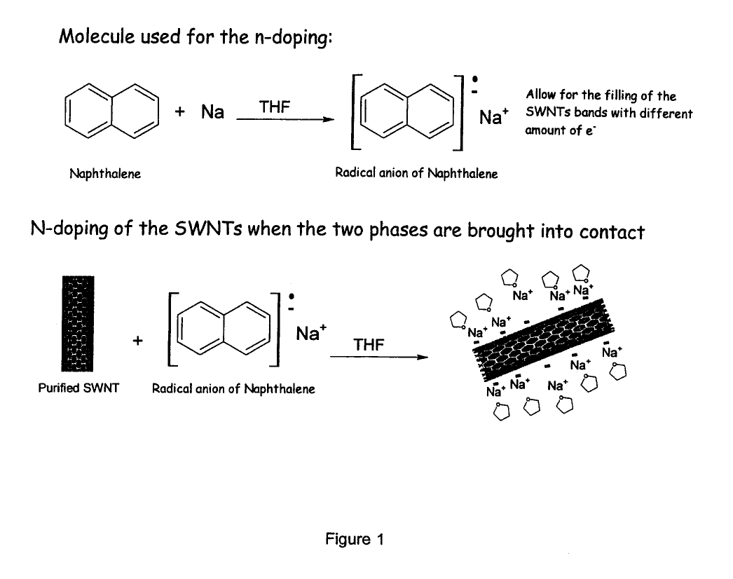

In another embodiment, one operates as indicated above, and uses about 390 mg of naphthalene, about 120 mg of sodium metal, and about 220 mg of CNTs. The sodium naphthalene salt and the CNTs are stirred for about 15 hours. This reaction scheme is shown in FIG. 1.

The reduced CNTs can then be functionalized using the processes described more fully below.

### Chemical Functionalization

Following the dispersion of the CNT salt, chemical functionalization can occur readily using functionalization processes described in the prior art that have been applied to neutral CNTs. For example and without being limited thereto, chemical functionalization can occur using oxidizing agents, thermally unstable, radical producing species such as ozone, DMSO, peroxides and other radical producing species, azo compounds, diazonium compounds, and stable radicals such as NO (nitric oxide). Reactions of most of these species with neutral CNT have been demonstrated, for example, by Khabashesku et al, U.S. Patent Application Publication No. 2004/0223900; Huang et al., U.S. Patent Application Publication No. 2005/0229334; Tour et al., U.S. Patent Application Publication No. 2004/0071624; Peng et al., J. Am. Chem. Soc., 125, 15174 (2003); and Umek et al., Chem. Mater., 15,4751 (2003), each disclosure of which is incorporated by reference. It has been demonstrated by these prior art processes that such functionalization with neutral CNTs requires several hours, even days, to achieve a sufficient functionalization level. Such functionalization applied to the dispersed CNT salt described can reduce reaction times. This functionalization is applicable to SWNT, DWNT, and MWNT salts. In the case of DWNTs and MWNTs, the outer sidewall can be functionalized in the same manner as that of the single-wall of a SWNT.

For example, using the chemical functionalization procedures described in Huang et al., U.S. Patent Application Publication No. 2005/0229334, the CNT salt may be similarly chemically functionalized.

The chemical functionalization of the carbon nanotube sidewall results in functional groups, including but not limited to, —COOH, —PO₄⁻, —SO₃⁻, —SO₃H, —SH, —NH₂, tertiary amines, quaternary amines, —CHO, —OH, alkyl, alkenyl, alkynyl, cycloalkyl, heterocyclyl, cycloalkenyl, alkoxy, alkanoyl, acyl, aryl, and/or heteroaryl.

The following terms are meant to encompass unsubstituted or substituted.

“Alkyl” means straight and branched carbon chains. Examples of such alkyl groups include, but are not limited to, methyl, ethyl, isopropyl, tert-butyl, neopentyl, and n-hexyl. The alkyl groups can also have at least one heteroatom selected from O, S, or N. The alkyl groups can be substituted if desired, for instance with groups such as hydroxy, amino, alkylamino, and dialkylamino, halo, trifluoromethyl, carboxy, nitro, and cyano, but no to be limited thereto.

“Alkenyl” means straight and branched hydrocarbon radicals having at least one double bond, conjugated and/or unconjugated, and includes, but is not limited to, ethenyl, 3-buten-1-yl, 2-ethenylbutyl, 3-hexen-1-yl, and the like. The alkenyl can also have at least one heteroatom selected from O, S, or N.

“Alkyny” means straight and branched hydrocarbon radicals having at least one triple bond, conjugated and/or unconjugated, and includes, but is not limited to, ethynyl, 3-butyn-1-yl, propynyl, 2-butyn-1-yl, 3-pentyn-1-yl, and the like. The alkynyl can also have at least one heteroatom selected from O, S,or N.

“Cycloalkyl” means a monocyclic or polycyclic hydrocarbyl group such as, but not limited to, cyclopropyl, cycloheptyl, cyclooctyl, cyclodecyl, cyclobutyl, adamantyl, norpinanyl, decalinyl, norbomyl, cyclohexyl, and cyclopentyl. Such groups can be substituted with groups such as hydroxy, keto, and the like. Also included are rings in which heteroatoms can replace carbons. Such groups are termed “heterocyclyl”, which means a cycloalkyl group also bearing at least one heteroatom selected from O, S. or N.

“Cycloalkenyl” means a monocyclic or polycyclic hydrocarbyl group having at least one double bond, conjugated and/or unconjugated, such as, 2004/0223900, the CNT salt may be similarly chemically functionalized. For instance, the CNT salt can be reacted with the carbon-centered generated free radicals of acyl peroxides. This allows for the chemical attachment of a variety of functional groups to the wall or end cap of carbon nanotubes through covalent carbon bonds. Carbon-centered radicals generated from acyl or aroyl peroxides can have terminal functional groups that provide sites for further reaction with other compounds. Organic groups with terminal carboxylic acid functionality can be converted to an acyl chloride and further reacted with an amine to form an amide or with a diamine to form an amide with terminal amine, for example. The reactive functional groups attached to the nanotubes provide improved solvent dispersibility and provide reaction sites for monomers for incorporation in polymer structures. The nanotubes can also be functionalized by generating free radicals from organic sulfoxides.

The decomposition of acyl or aroyl peroxides is used to generate carbon-centered free radicals, which non-destructively add organic groups through a carbon linkage to the CNT salt. Acyl or aroyl peroxides, or alternatively, diacyl or diaroyl peroxides, have the chemical formula. R—C(O)O—O(O)C—R′. The O—O bond is very weak and under suitable conditions, the O—O bond can readily undergo bond homolysis to form an intermediate carboxyl radical which decarboxylates to produce carbon dioxide and carbon-centered radicals, such as —R, —R′, or a combination thereof. The R and R′ groups can be the same or different. The R and R′ can be any suitable group, for example, and without being limited thereto, alkyl, alkenyl, alkynyl, cycloalkyl, heterocyclyl, cycloalkenyl, aryl, and/or heteroaryl groups; and any of the like. In addition, the R and R′ groups can have terminal functional groups and contain heteroatoms, other than carbon and hydrogen. Acyl and aroyl peroxides are conveniently and economically available, or can be synthesized, with a wide variety of R and R′ groups.

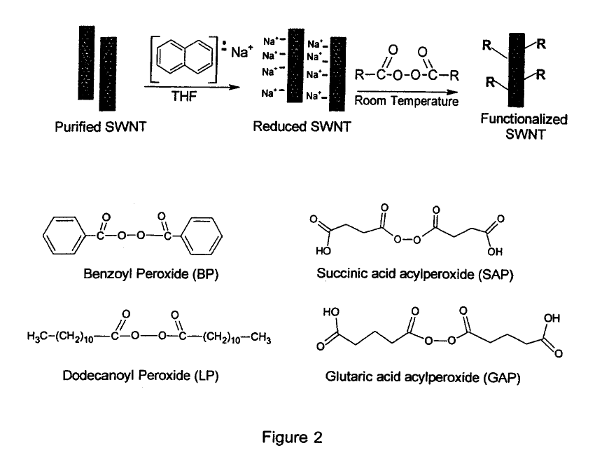

As shown in FIG. 2, a group, such as a phenyl group, can be bonded to the CNT salt using phenyl groups generated by the decomposition of the aroyl peroxide, for example, benzoyl peroxide. Other acyl and/or aroyl peroxides can also be used such as, and without being limited thereto, lauroyl but not limited to, cyclopropenyl, cycloheptenyl, cyclooctenyl, cyclodecenyl, and cyclobutenyl. Such groups can be substituted with groups such as hydroxy, keto, and the like.

“Alkoxy” refers to the alkyl groups mentioned above bound through oxygen, examples of which include, but are not limited to, methoxy, ethoxy, isopropoxy, tert-butoxy, and the like.

“Alkanoyl” groups are alkyl linked through a carbonyl. Such groups include, but are not limited to, formyl, acetyl, propionyl, butyryl, and isobutyryl.

“Acyl” means an R group that is an alkyl or aryl group bonded through a carbonyl group, i.e., R—C(O)—. For example, acyl includes, but is not limited to, a C1-C6 alkanoyl, including substituted alkanoyl. Typical acyl groups include acetyl, benzoyl, and the like.

The terms “aryl” or “aromatic” refers to unsubstituted and substituted monoaromatic or polyaromatic groups that may be attached together in a pendent manner or may be fused, which includes, but is not limited to, phenyl, naphthyl, tetrahydro-naphthyl, indanyl, biphenyl, phenanthryl, anthryl or acenaphthyl. The “aryl” group may have 1 to 3 substituents such as alkyl, hydroxyl, halo, haloalkyl, nitro, cyano. alkoxy, alkylamino and the like.

The terms “heteroaryl” or “heteroaromatic” refers to unsubstituted and substituted monoaromatic or polyaromatic groups having at least one heteroatom selected from O, S, or N, which includes, but is not limited to, indazolyl, pyrrolyl, pyrazolyl, imidazolyl, thiazolyl, thiophenyl, and the like.

CNT's may be functionalized using free radical organic initiators, such as azo-initiators. The azo compound forms free radicals via the loss of nitrogen, the resultant radicals can couple to the CNT salt described herein. Such compounds can result in functional groups, including but not limited to, alkyl groups such as saturated aliphatic chain(s); alkenyl groups such as unsaturated chain(s) and conjugated chain(s); cyclic group(s); and/or aromatic group(s) and any of the like. The chain(s) can be of any suitable length, including polymer chain(s).

In other examples, using the chemical functionalization procedures described in Khabashesku et al., U.S. Patent Application Publication No. peroxide, succinic acid acylperoxide (SAP), glutaric acid acylperoxide (GAP). The procedures for attaching such groups to the CNT salt comprises making a dispersion of the CNT salt in a suitable solvent, such as THF, and adding acyl and/or aroyl peroxide to the dispersion and agitating the mixture (e.g. stirring, sonicating, etc.). The mixture is at room temperature and mixed for a time effective to decompose the peroxide, generate free carbon-entered radicals and bond the free radicals to the sidewalls of the CNT salt.

Examples of suitable acyl peroxides of the form R—C(O)O—O(O)C—R′, wherein the R and R′ are organic groups that can be the same or different and can include, but are not limited to, acetyl peroxide, n-butyryl peroxide, secbutyryl peroxide, t-butyryl peroxide, t-pentoyl peroxide, iso-valeryl peroxide, furoyl peroxide, palmitoyl peroxide, decanoyl peroxide, lauroyl peroxide, diisopropyl peroxydicarbonate and butylperoxyisopropyl carbonate. The R or R′ group can comprise a normal, branched or cyclic alkyl group wherein the number of carbons can range from one to about 30, and typically, in the range of about 8 to about 20. The R or R′ group can contain one or more cyclic rings, examples of which are trans4-butylcyclohexanoyl peroxide, trans-4-cyclohexanecarbonyl peroxide and cyclohexyl peroxydicarbonate, cyclopropanoyl peroxide, cyclobutanoyl peroxide and cyclopentanoyl peroxide. The acyl peroxides can contain heteroatoms and functional groups, such as bromobutyryl peroxide, (CCl₃CO₂)₂, (CF₃CO₂)₂, (CCl₃CO₂)₂, (RO(CH₂)ₙCO₂)₂, (RCH═CR′CO₂)₂, RC═CCO₂)₂, and (N═C(CH₂)ₙCO₂)₂, where n=1-3.

Examples of suitable aroyl peroxides of the form R—C(O)O—O(O)C—R′, wherein the R and R′ are organic groups that can be the same or different and can include, but are not limited to, cinnamoyl peroxide, bis(p-methoxybenzoyl)peroxide, p-monomethoxybenzoyl peroxide, bis(o-phenoxybenzoyl)peroxide, acetyl benzoyl peroxide, t-butyl peroxybenzoate, diisopropyl peroxydicarbonate, cyclohexyl peroxydicarbonate, benzoyl phenylacetyl peroxide, and butylperoxyisopropyl carbonate. The aroyl peroxide can also include heteroatoms, such as in p-nitrobenzoyl peroxide, p-bromobenzoyl, p-chlorobenzoyl peroxide, and bis(2,4-dichlorobenzoyl)peroxide. The aroyl peroxide can also have other substituents on one or more aromatic rings, such as in p-methylbenzoyl peroxide, p-methoxybenzoyl peroxide, o-vinylbenzoyl benzoyl peroxide, and exo- and endo-norbornene-5-carbonyl peroxide. The aromatic ring substitutions of the various groups and heteroatoms can also be in other positions on the ring, such as the ortho, meta or para positions. The aroyl peroxide can also be an asymmetric peroxide and include another organic group that can be an alkyl, cyclic, aromatic, or combination thereof.

Alkyl groups terminated with the carboxylic acid functionality, as shown for example in FIG. 2, can be attached to the sidewalls of the CNT. FIG. 2 shows an embodiment wherein a dicarboxylic acid acyl peroxide such as GAP or SAP, liberates CO₂ and generates a carbon-centered free radical which bonds to the sidewall of the CNT salt to form sidewall functionalized CNTs with organic groups having terminal carboxylic acid groups.

Functionalized CNTs with sidewall alkyl groups having terminal carboxylic acid functionality can further be reacted to yield nanotubes with other reactive functionality. For example, amide derivatives can be made by reacting the carboxylic acid functionality with a chlorinating agent, such as thionyl chloride, and subsequently with an amine compound. Other possible chlorinating agents, include, but are not limited to phosphorous trichloride, phosphorous pentachloride, and oxalyl chloride. To give the CNT side group a terminal amine, a diamine can be used. Examples of suitable diamines are ethylene diamine, 4,4′-methylenebis(cyclohexylamine), propylene diamine, butylene diamine, hexamethylene diamine and combinations thereof.

For solution phase reactions, the acyl and/or aroyl peroxide is added to the dispersion of the CNT salt; the CNT salt is dispersed in any suitable polar organic solvent(s). For example and without being limited thereto, the solvent can be pyridine, tetrahydrofuran (THF), ethers, 1,2-dimethoxyethane (DME), and/or toluene. The mixture can be maintained at room temperature under an inert atmosphere and can be completed within about 30 minutes.

After the CNT functionalization reaction is complete, the functionalized CNT can be isolated from unreacted peroxides and by-products by washing with solvent. For example, sidewalled-functionalized SWNT can be purified by washing with a solvent, such as chloroform. The nanotubes can then be dried, such as in a vacuum oven.

Methyl radicals can also be generated from dimethyl sulfoxide (DMSO) by the method of Minisci (see Fontana et al., Tetrahed, Lett. 29, 1975-1978 (1988). “Minisci”, incorporated herein by reference) by reaction with hydroxyl radicals. A convenient source of hydroxyl radicals can be generated using Fenton's reagent, which includes hydrogen peroxide and a divalent iron catalyst. The methyl radicals generated from the dimethyl sulfoxide and hydroxyl radicals can bond to the negatively charged CNTs to form sidewall methylated carbon nanotubes.

Alkyl and aryl radicals can be generated using the Minisci method using sulfoxides with various alkyl and/or aryl groups. In this embodiment, sulfoxides, which have the form R—S(O)—R′, where —R and —R′ can be the same or different, can also be used to generate various carbon radicals. The R groups can be alkyl or aromatic or a combination thereof. This process offers another route to other free radicals and another embodiment for adding functional groups to the CNT salt sidewall. The R or R′ group generally can comprise a number of carbons in the range of 1 and about 30.

The degree of functionalization of the CNT will depend on various factors, including, but not limited to, the type and structure of side group, steric factors, the desired level for an intended end-use, and the functionalization route and conditions. The generally accepted maximum degree of functionalization of a CNT, in particular a SWNT, is 1 functional group per 100 nanotube carbons.

### Combination of Dispersion and Functionalization

In an embodiment, the process comprises dispersing a CNT salt; and functionalizing the CNT salt.

Formation of the dispersion of the CNT salt can be achieved using, for example, the procedures described above under the heading “dispersion”. The negatively charged CNT of the CNT salt dispersion is chemically functionalized using, for example and without being limited thereto, any of the procedures described above under the heading “chemical functionalization” that will provide functionalization.

In embodiments, the CNTs of the CNT salt dispersion are negatively charged CNTs. In further embodiments, chemical functionalization of the negatively charged CNTs occurs through radical producing species.

In certain embodiments, the process and materials of the invention can reduce reaction times from days and hours to minutes, producing chemically functionalized CNTs at the single tube level. Similarly, this can also be achieved with DWNTs and MWNTs.

The process of dispersing the CNT salt and chemical functionalization of the CNT salt can be achieved in a single-pot process; can provide covalently functionalized CNTs; can be efficient and take place within minutes; can be conducted at room temperature; and can control the degree and type of functionalization.

Chemical functionalization of the process occurs at a temperature that initiates chemical functionalization. In certain cases, the temperature can even be about room temperature.

In another embodiment, the CNT salt dispersion is formed using the processes described in Penicaud et al. and described in International Patent Application No. WO 2005/073127 and the J. Amer. Chem. Soc., 127, 8 (2005) that incorporate alkali salt(s). Chemical functionalization of the CNT salt is done using any of the procedures described above, for example, under the heading “chemical functionalization” that will provide functionalization. In specific embodiments. the process of the invention is a single pot process. For example, the CNT salt formation and chemical functionalization takes place in a single flask, which is a cost-effective and time-effective way of providing side-wall chemical functionalization. Such an embodiment of the process provides a process useful to rapidly and efficiently de-bundle and functionalize CNTs. Chemical functionalization of SWNTs is needed for the integration and use of CNTs in advanced materials.

Functionalized CNTs can be used as starting material for another cycle of functionalization (e.g. to achieve multi-level functionalization). For example, instead of using an unfunctionalized CNT salt dispersion, a functionalized CNT salt dispersion is used and further chemically functionalized as discussed herein. This increases the degree of functionalization of CNTs.

The above disclosure generally describes the present invention. A more complete understanding can be obtained by reference to the following specific Examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Starting Materials

### Preparation of SWNT, DWNT and MWNT

The SWNT was made using the process described in Kingston et al., Carbon, 42, 1657 (2004). SWNT can also be obtained from companies such as Carbolex Inc. (Lexington, Ky., U.S.A.), Carbon Nanotechnologies Inc. (Houston, Tex. U.S.A.), Thomas Swan & Co. Ltd. (Crookhall, Consett, U.K.), Nanocyl (Rockland, Mass., U.S.A.) and Cheap Tubes, Inc. (Brattleboro, Vt., U.S.A.).

The DWNT can be obtained from Carbon Nanotechnologies Inc. (Houston, Tex., U.S.A.) and Nanocyl (Rockland, Mass., U.S.A.).

The MWNT can be obtained from Nanocyl (Rockland, Mass., U.S.A.) and Cheap Tubes, Inc. (Brattleboro, Vt., U.S.A.).

### Preparation of Glutaric Acid Acylperoxide (GAP)

About 10 g of glutaric anhydride fine powder (Aldrich) was added to about 20 mL of an ice cold solution of 8% hydrogen peroxide. The mixture was stirred for about 1 hour and then filtered using a 5 μm polycarbonate filter. The resulting glutaric acid acylperoxide was washed with cold water, air-dried for about 10 minutes and then dried under vacuum at room temperature for about 24 hours.

### Preparation of Succinic Acid Acylperoxide (SAP)

About 10 g of succinic anhydride fine powder (Aldrich) was added to about 20 mL of an ice cold solution of 8% hydrogen peroxide. The mixture was stirred for about 1 hour and then filtered using a 5 μm polycarbonate filter. The resulting succinic acid acylperoxide was washed with cold water, air-dried for about 10 minutes and then dried under vacuum at room temperature for about 24 hours.

### Examples with SWNT

Dispersion and Chemical Functionalization using SWNT

The reaction was done under inert atmosphere and is shown in FIG. 1 and FIG. 2 (for (a)-(d) below). The functionalization procedure can take place in one flask.

### SWNT Salt

About 24 mg (2 mM) of purified SWNT was suspended, for about 30 minutes, in 20 mL of dry THF, using an ultrasonic tip. About 16 mg (0.7 mM) of sodium and about 90 mg (0.7 mM) of naphthalene were added to the suspension. A green mixture was formed and the suspension stirred overnight, providing the SWNT salt (see FIG. 1).

This Reaction is followed by one of the subsequent procedures (a) to (e):
a) Functionalization using Dibenzoyl Peroxide

About 2 mM of dibenzoyl peroxide (obtained from Aldrich) was dissolved in 15 mL of toluene and added to the SWNT salt. The reaction mixture was stirred at room temperature for about 30 minutes. The reaction mixture was filtered using a 3 μm pore size PTFE membrane (Millipore). The product was washed, sequentially, with toluene, THF, water and methanol. The functionalized SWNTs were repeatedly suspended in THF, then methanol and then DMF, using an ultrasonic bath. The suspensions were centrifuged and finally filtrated to recover the product which was washed with acetone and dried under vacuum at 80° C.

b) Functionalization using Lauroyl Peroxide

About 2 mM of lauroyl peroxide (obtained from Aldrich) was dissolved in 15 mL of toluene and added to the SWNT salt. The reaction mixture was stirred at room temperature for about 30 minutes. The reaction mixture was filtered using a 3 μm pore size PTFE membrane (Millipore). The product was washed, sequentially, with toluene, THF, water and methanol. The functionalized SWNTs were repeatedly suspended in THF, then methanol and then DMF, using an ultrasonic bath. The suspensions were centrifuged and finally filtrated to recover the product which was washed with acetone and dried under vacuum at 80° C.

c) Functionalization using Glutaric Acid Acylperoxide (GAP) About 2 mM of glutaric acid acylperoxide (prepared as described above) was added directly to the SWNT salt. The reaction mixture was stirred at room temperature for about 30 minutes. The reaction mixture was filtered using a 3 μm pore size PTFE membrane (Millipore). The product was washed, sequentially, with toluene, THF, water and methanol. The functionalized SWNTS were repeatedly suspended in THF, then methanol and then DMF, using an ultrasonic bath. The suspensions were centrifuged and finally filtrated to recover the product which was washed with acetone and dried under vacuum at 80° C.
d) Functionalization using Succinic Acid Acylperoxide (SAP)

About 2 mM of succinic acid acylperoxide (prepared as described above) was added directly to the SWNT salt. The reaction mixture was stirred at room temperature for about 30 minutes. The reaction mixture was filtered using a 3 μm pore size PTFE membrane (Millipore). The product was washed. sequentially, with toluene, THF, water and methanol. The functionalized SWNTs were repeatedly suspended in THF, then methanol and then DMF, using an ultrasonic bath. The suspensions were centrifuged and finally filtrated to recover the product which was washed with acetone and dried under vacuum at 80° C.

e) Functionalization using Azo Compounds

About 2 mM of 2,2′-azobis(4-cyanovaleric acid) is added directly to the SWNT salt. The reaction is stirred at a temperature to form the free radicals of the azo compound and yield the functionalized product The reaction mixture containing the product is filtered using a 3 μm pore size PTFE membrane (Millipore). The product is washed, sequentially, with toluene, THF, water and methanol. The functionalized SWNTs are repeatedly suspended in THF, then methanol and then DMF, using an ultrasonic bath. The suspensions are centrifuged and are finally filtrated to recover the product which is washed with acetone and is dried under vacuum at 80° C.

f) Functionalization using DMSO

About 155 mg of purified SWNT was suspended in 150 mL of dry THF and sonicated using an ultrasonic tip for about 30 minutes. About 146 mg of small pieces of sodium and about 964 mg of naphthalene were added to the suspension. The mixture was stirred overnight at room temperature. The resulting green mixture was centrifuged at 5000 RPM for 30 minutes, and then the precipitate was washed once with dry THF and centrifuged again to provide the SWNT salt (see FIG. 1).

About 30 mL of dry DMSO (dried with molecular sieve 4 Å) was added to the SWNT salt under inert atmosphere. The mixture was shaken by hand. Gases evolved immediately indicating a rapid reaction. After about 10 minutes the mixture was centrifuged, and the precipitate was washed with THF. After drying under vacuum at about 95° C., the sample was analyzed using Raman spectroscopy. A substantial increase in the D-band near 1350 cm⁻¹ was observed indicating side-wall functionalization. In addition, the solubility of the precipitate was significantly increased in DMSO compared with its starting material (neutral SWNTS).

### Examples with DWNT

These DWNT examples provide a degree of functionalization of the DWNT that is slightly more than the degree of functionalization of the SWNT of the above-identified examples.

### Dispersion and Chemical Functionalization Using DWNT

The reaction is done under inert atmosphere. The functionalization procedure can take place in one flask.

### DWNT Salt

About 24 mg (2 mM) of purified DWNT is suspended, for about 30 minutes, in 20 mL of dry THF, using an ultrasonic tip. About 16 mg (0.7 mM) of sodium and about 90 mg (0.7 mM) of naphthalene are added to the suspension. The suspension is stirred overnight, providing the DWNT salt

This reaction is followed by one of the subsequent Procedures (a) to (e):
a) Functionalization using Dibenzoyl Peroxide

About 2 mM of dibenzoyl peroxide (obtained from Aldrich) is dissolved in 15 mL of toluene and is added to the DWNT salt. The reaction mixture is stirred at room temperature for about 30 minutes. The reaction mixture is filtered using a 3 μm pore size PTFE membrane (Millipore). The product is washed, sequentially, with toluene, THF, water and methanol. The functionalized DWNTs are repeatedly suspended in THF, then methanol and then. DMF, using an ultrasonic bath. The suspensions are centrifuged and finally filtrated to recover the product which is washed with acetone and is dried under vacuum at 80° C.

b) Functionalization using Lauroyl Peroxide

About 2 mM of lauroyl peroxide (obtained from Aldrich) is dissolved in 15 mL of toluene and is added to the DWNT salt. The reaction mixture is stirred at room temperature for about 30 minutes. The reaction mixture is filtered using a 3 μm pore size PTFE membrane (Millipore). The product is washed, sequentially, with toluene, THF, water and methanol. The functionalized DWNTs are repeatedly suspended in THF, then methanol and then DMF, using an ultrasonic bath. The suspensions are centrifuged and finally filtrated to recover the product which is washed with acetone and is dried under vacuum at 80° C.

c) Functionalization using Glutaric Acid Acylperoxide (GAP)

About 2 mM of glutaric acid acylperoxide (prepared as described above) is added directly to the DWNT salt. The reaction mixture is stirred at room temperature for about 30 minutes. The reaction mixture is filtered using a 3 μm pore size PTFE membrane (Millipore). The product is washed, sequentially, with toluene, THF, water and methanol. The functionalized DWNTs are repeatedly suspended in THF, then methanol and then DMF, using an ultrasonic bath. The suspensions are centrifuged and finally filtrated to recover the product which is washed with acetone and is dried under vacuum at 80° C.

d) Functionalization using Succinic Acid Acylperoxide (SAP)

About 2 mM of succinic acid acylperoxide (prepared as described above) is added directly to the DWNT salt. The reaction mixture is stirred at room temperature for about 30 minutes. The reaction mixture is filtered using a 3 μm pore size PTFE membrane (Millipore). The product is washed, sequentially, with toluene, THF, water and methanol. The functionalized DWNTs are repeatedly suspended in THF, then methanol and then DMF, using an ultrasonic bath. The suspensions are centrifuged and finally filtrated to recover the product which is washed with acetone and is dried under vacuum at 80° C.

e) Functionalization using Azo Compounds

About 2 mM of 2,2′-azobis(4-cyanovaleric acid) is added directly to the DWNT salt. The reaction is stirred at a temperature to form the free radicals of the azo compound and yield the functionalized product. The reaction mixture containing the product is filtered using a 3 μm pore size PTFE membrane (Millipore). The product is washed, sequentially, with toluene, THF, water and methanol. The functionalized DWNTs are repeatedly suspended in THF, then methanol and then DMF, using an ultrasonic bath. The suspensions are centrifuged and are finally filtrated to recover the product which is washed with acetone and is dried under vacuum at 80° C.

f) Functionalization using a DMSO

About 155 mg of purified DWNT. is suspended in 150 mL of dry THF and is sonicated using an ultrasonic tip for about 30 minutes. About 146 mg of small pieces of sodium and about 964 mg of naphthalene are added to the suspension. The mixture is stirred overnight at room temperature. The resulting green mixture is centrifuged at 5000 RPM for 30 minutes, and then the precipitate is washed once with dry THF and is centrifuged again to provide the DWNT salt.

About 30 mL of dry DMSO (dried with molecular sieve 4 Å) is added to the DWNT salt under inert atmosphere. The mixture is shaken by hand. Gases evolve immediately indicating a rapid reaction. After about 10 minutes the mixture is centrifuged, and the precipitate is washed with THF and is dried under vacuum at about 95° C.

### Examples with MWNT

These MWNT examples provide a degree of functionalization of the MWNT that is more than the degree of functionalization of the SWNT of the above-identified examples.

### Dispersion and Chemical Functionalization Using MWNT

The reaction is done under inert atmosphere. The functionalization procedure can take place in one flask.

### MWNT Salt

About 24 mg (2 mM) of purified MWNT is suspended, for about 30 minutes, in 20 mL of dry THF, using an ultrasonic tip. About 16 mg (0.7 mM) of sodium and about 90 mg (0.7 mM) of naphthalene are added to the suspension. The suspension is stirred overnight, providing the MWNT salt.

This reaction is followed by one of the subsequent procedures (a) to (e):
a) Functionalization using Dibenzoyl Peroxide

About 2 mM of dibenzoyl peroxide (obtained from Aldrich) is dissolved in 15 mL of toluene and is added to the MWNT salt The reaction mixture is stirred at room temperature for about 30 minutes. The reaction mixture is filtered using a 3 μm pore size PTFE membrane (Millipore). The product is washed, sequentially, with toluene, THF, water and methanol. The functionalized MWNTs are repeatedly suspended in THF, then methanol and then DMF, using an ultrasonic bath. The suspensions are centrifuged and finally filtrated to recover the product which is washed with acetone and is dried under vacuum at 80° C.

b) Functionalization using Lauroyl Peroxide

About 2 mM of lauroyl peroxide (obtained from Aldrich) is dissolved in 15 mL of toluene and is added to the MWNT salt. The reaction mixture is stirred at room temperature for about 30 minutes. The reaction mixture is filtered using a 3 μm pore size PTFE membrane (Millipore). The product is washed, sequentially, with toluene, THF, water and methanol. The functionalized MWNTs are repeatedly suspended in THF, then methanol and then DMF, using an ultrasonic bath. The suspensions are centrifuged and finally filtrated to recover the product which is washed with acetone and is dried under vacuum at 80° C.

c) Functionalization using Glutaric Acid Acylperoxide (GAP)

About 2 mM of glutaric acid acylperoxide (prepared as described above) is added directly to the MWNT salt. The reaction mixture is stirred at room temperature for about 30 minutes. The reaction mixture is filtered using a 3 μm pore size PTFE membrane (Millipore). The product is washed, sequentially. with toluene, THF, water and methanol. The functionalized MWNTs are repeatedly suspended in THF, then methanol and then DMF, using an ultrasonic bath. The suspensions are centrifuged and finally filtrated to recover the product which is washed with acetone and is dried under vacuum at 80° C.

d) Functionalization using Succinic Acid Acylperoxide (SAP)

About 2 mM of succinic acid acylperoxide (prepared as described above) is added directly to the MWNT salt. The reaction mixture is stirred at room temperature for about 30 minutes. The reaction mixture is filtered using a 3 μm pore size PTFE membrane (Millipore). The product is washed, sequentially, with toluene, THF, water and methanol. The functionalized MWNTs are repeatedly suspended in THF, then methanol and then DMF, using an ultrasonic bath. The suspensions are centrifuged and finally filtrated to recover the product which is washed with acetone and is dried under vacuum at 80° C.

a) Functionalization using Azo Compounds

About 2 mM of 2,2′-azobis(4cyanovaleric acid) is added directly to the MWNT salt. The reaction is stirred at a temperature to form the free radicals of the azo compound and yield the functionalized product. The reaction mixture containing the product is filtered using a 3 μm pore size PTFE membrane (Millipore). The product is washed, sequentially, with toluene, THF, water and methanol. The functionalized MWNTs are repeatedly suspended in THF, then methanol and then DMF, using an ultrasonic bath. The suspensions are centrifuged and are finally filtrated to recover the product which is washed with acetone and is dried under vacuum at 80° C.

f) Functionalization using DMSO

About 155 mg of purified MWNT is suspended in 150 mL of dry THF and is sonicated using an ultrasonic tip for about 30 minutes. About 146 mg of small pieces of sodium and about 964 mg of naphthalene are added to the suspension. The mixture is stirred overnight at room temperature. The resulting green mixture is centrifuged at 5000 RPM for 30 minutes, and then the precipitate is washed once with dry THF and is centrifuged again to provide the MWNT salt.

About 30 mL of dry DMSO (dried with molecular sieve 4 Å) is added to the MWNT salt under inert atmosphere. The mixture is shaken by hand. Gases evolve Immediately indicating a rapid reaction. After about 10 minutes the mixture is centrifuged, and the precipitate is washed with THF and is dried under vacuum at about 95° C.

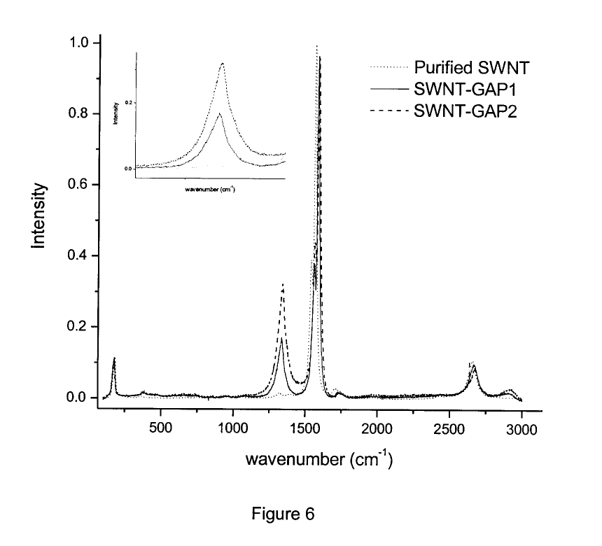

The resultant functionalized CNTs resulting from the above examples can be used as a starting material for another cycle of functionalization (e.g. multi-level functionalization). This increases the degree of functionalization, as confirmed by the increase in the D-band (SWNT-GAP2 of FIG. 6 discussed more fully below).

### Characterization of Resultant Functionalized SWNTs

Raman spectroscopy is a sensitive tool to analyze CNTs. Of particular interest here is the 1350 cm⁻¹ Stoke shift region of the Raman spectrum, known as the D-band (D stands for disorder). It indicates the disorder state of the graphene network forming the CNT. In the pristine CNT, this band should preferably be very small. Side-wall chemical functionalization occurs by disrupting the graphene network. For example, it causes a change from sp² hybridization to sp³ hybridization. When this occurs, the D-band will increase. It is recognized that an increase in the D-band is a good indicator that side-wall functionalization has taken place. Additional evidence is provided by a change in solubility, which was noticed after functionalization.

Functionalization using Dibenzoyl Peroxide (BP)

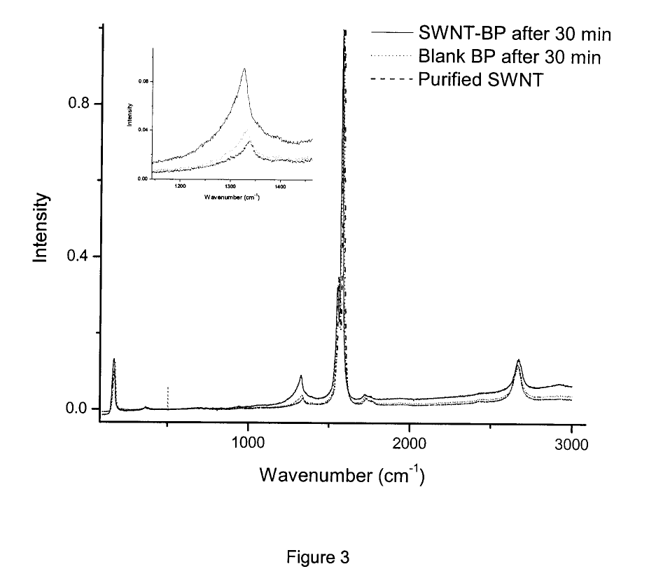

The Raman spectrum (SWNT-BP) for the embodiment of (a) for SWNT, utilizing dibenzoyl peroxide (BP) and the SWNT salt, is shown in FIG. 3. The spectrum is compared with the results of a “blank test” in which the same experimental conditions were used except with neutral SWNTs (Blank BP=neutral SWNT+BP). The spectra are also compared with the spectrum of pristine SWNT (Purified SWNT). As can be seen, no or very little functionalization occurs with the neutral SWNTs. When the SWNT salt was used, the increase in the D-band intensity shows that side-walled functionalization has occurred.

Functionalization using Lauroyl Peroxide (LP)

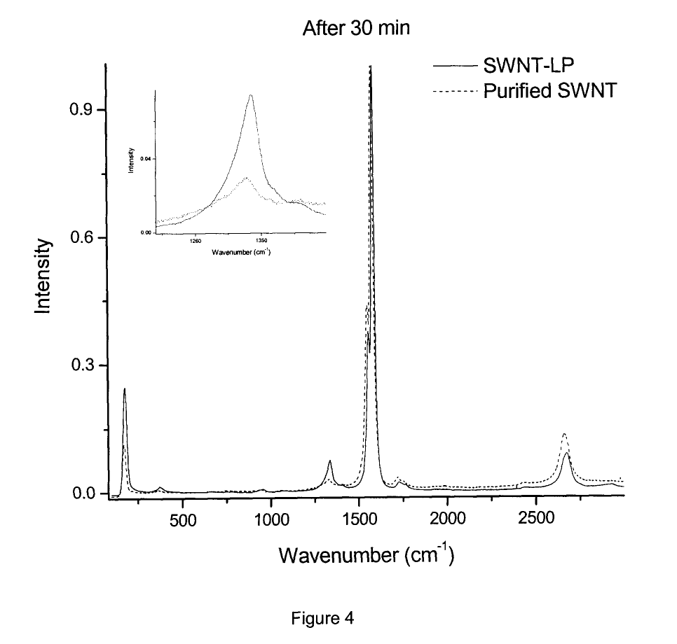

The Raman spectrum (SWNT-LP after 30 min) for the embodiment of (b) for SWNT, utilizing lauroyl peroxide (LP) and the SWNT salt, is shown in FIG. 4. The spectrum is compared with the spectrum of pristine SWNT (Purified SWNT). In this case, the increase in the D-band intensity shows that side-walled functionalization has occurred after about 30 minutes at room temperature.

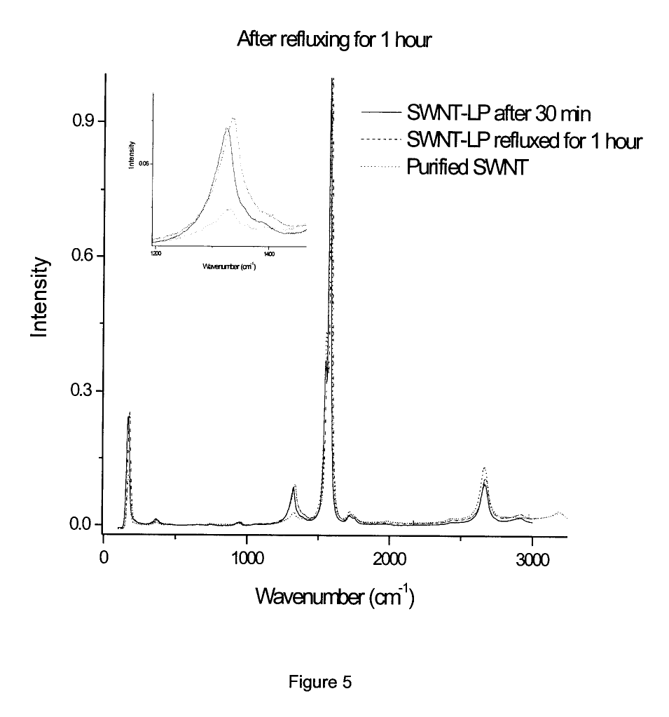

The experiment with lauroyl peroxide was continued. After 30 minutes of functionalization at room temperature, the reaction mixture was brought to reflux for one hour (SWNT-LP refluxed for 1 hour). As shown in FIG. 5, the D-band is no more intense than after reaction for about 30 minutes at room temperature. This indicates that the reaction occurs readily and rapidly without the need to supply heat and is substantially complete within about 30 minutes.

Functionalization using Glutaric Acid Acylperoxide (GAP)

The Raman spectrum (SWNT-GAP1 ) for the embodiment of (c) for SWNT, utilizing glutaric acid acylperoxide (GAP1) and the SWNT salt is shown in FIG. 6. Similar results were obtained with succinic acid acylperoxide. The spectrum is compared with the spectrum of pristine SWNT (Purified SWNT). As can be seen, the increase in the D-band intensity shows that side-walled functionalization has occurred after about 30 minutes at room temperature (SWNT-GAP1).

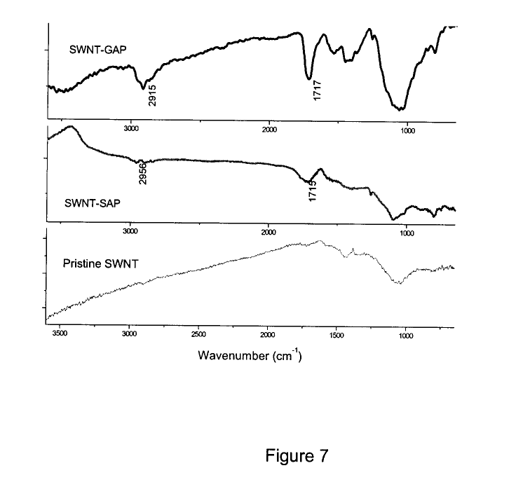

The resultant functionalized CNT, specifically SWNT-GAP1, can be used as starting material for another cycle of reaction (SWNT-GAP2). This allowed for an increase in the degree of functionalization, as can be confirmed by the increase in the D-band (SWNT-GAP2). Infrared spectroscopy was used to obtain information about the functional groups connected to the CNT sidewall. As is shown in FIG. 7, the infrared spectrum of pristine SWNTs are featureless, however, in the case of SWNT functionalized with glutaric (SWNT-GAP) and succinic (SWNT-SAP) acid acylperoxide, the peak at 1715 and 1717 cm⁻¹ region can be assigned to the carbonyl stretching mode, while the peaks in the 3000-2800 cm⁻¹ region can be attributed to the C—H stretching. The peaks in the 1560-1550 cm⁻¹ region are attributed to C═C stretching mode activated by sidewall attachment.

To determine the total percentage of carboxylic acid groups on the sidewall of the SWNT-GAP1 and SWNT-GAP2, purified SWNT and acid functionalized SWNT were titrated with NaHCO₃ solutions (Chem. Phys. Lett. 345, 25 (2001)). Quantitative results were attained by microwave assisted acidic leaching of sample material in 3M HNO₃ and determination of Na by Inductively Coupled Plasma-Atomic Emission Spectroscopy (ICP-AES). The results are shown in Table 1.

TABLE 1

| | |
|---|---|
| Sample ID | Na (ppm) |

| | | |
|---|---|---|
| SWNT-GAP1-Na | 16500 | ±2200 |
| SWNT-GAP2-Na | 17500 | ±2200 |
| Purified SWNT-Na | 3350 | ±200 |

These results indicate that I C% of functionalization (e.g. 1 out of every 100 carbon atoms forming the SWNT is functionalized) can be achieved after the second functionalization cycle.
Functionalization using DMSO

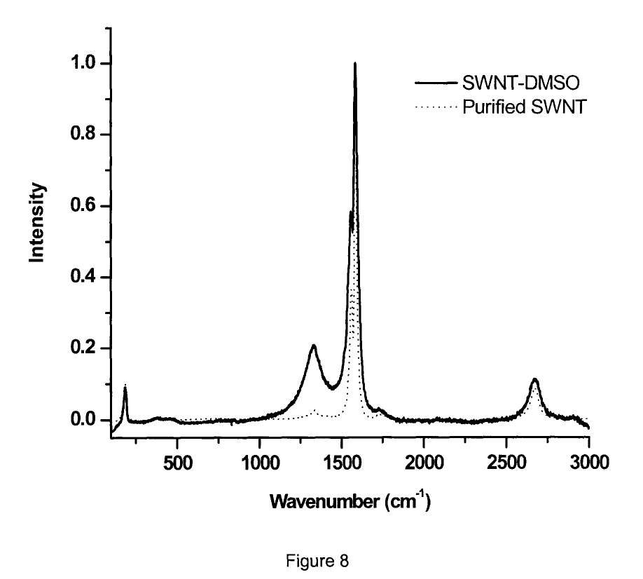

The Raman spectrum (SWNT-DMSO) for the embodiment of (f) for SWNT, utilizing DMSO and the SWNT salt, is shown in FIG. 8. The spectrum is compared with the spectrum of pristine SWNT (Purified SWNT). In this case, the increase in the D-band intensity shows that side-walled functionalization has occurred.

Comparison with the Approach of Umek et al. (Chem. Mat., 15, 4751 (2003)) and Margrave et al.l (J. Am. Chem. Soc. 125. 15174(2003), Incorporated Herein by Reference

Umek et al. have reported that dibenzoyl peroxide and lauroyl peroxide (the same two reagents used herein) can be used to functionalize the sidewall of SWNT. The reaction was conducted in toluene with neutral SWNT prior to the reaction with the peroxide. To obtain functionalization, the reaction mixture (neutral SWNT+peroxide in toluene) needed to be heated at 120° C. for 10 hours. In Margrave et al., the reaction took 10 days to be completed. In the process of the present invention, the SWNT salt, wherein the SWNT is negatively charged, is reacted with the peroxide and the functionalization reaction is substantially completed within about 30 minute.

When introducing elements disclosed herein, the articles “a”, “an”, “the”, and “said” are intended to mean that there are one or more of the elements. The terms “comprising”, “having”, “including” are intended to be open-ended and mean that there may be additional elements other than the listed elements.

All ranges given herein include the end of the ranges and also all the intermediate range points.

1. A process for chemically functionalizing carbon nanotubes, the process comprising:
dispersing carbon nanotube salt in a solvent; and
chemically functionalizing the carbon nanotube salt to provide chemically functionalized carbon nanotubes by reacting oxidizing agents or thermally unstable, radical producing species with the carbon nanotube salt.

2. The process of claim 1, wherein dispersing the carbon nanotube salt in the solvent comprises chemically reducing carbon nanotubes to the carbon nanotube salt, the carbon nanotube salt comprising negatively charged carbon nanotubes.

3. The process of claim 2, wherein chemically reducing the carbon nanotubes to the carbon nanotube salt comprises addition of a radical ion salt of formula A⁺B⁻ to the carbon nanotubes in the solvent, wherein A⁺ is a cation of an alkali metal and B⁻ is a radical anion of a polyaromatic compound.

4. The process of claim 2, wherein the alkali metal is lithium, potassium, and/or sodium.

5. The process of claim 3, wherein the alkali metal is lithium, potassium, or sodium.

6. The process of claim 3, wherein the polyaromatic compound is naphthalene and/or benzophenone.

7. The process of claim 1, wherein the solvent is a polar organic solvent.

8. The process of claim 7, wherein the polar organic solvent is pyridine, tetrahydrofuran, ethers, 1,2-dimethoxyethane, and/or toluene.

9. The process of claim 1, wherein the chemically functionalized carbon nanotubes comprise functional groups selected from —COOH, —PO₄⁻, —SO₃⁻, —SO₃H, —SH, —NH₂, tertiary amines, quaternary amines, —CHO, —OH, alkyl, alkenyl, alkynyl, cycloalkyl, heterocyclyl, cycloalkenyl, alkoxy, alkanoyl, acyl, aryl, and/or heteroaryl groups.

10. The process of claim 9, wherein the functional groups are alkyl or alkenyl groups.

11. (canceled)

12. The process of claim 1, wherein chemically functionalizing the carbon nanotube salt comprises reacting oxidizing agents with the carbon nanotube salt.

13. The process of claim 1, wherein chemically functionalizing the carbon nanotube salt comprises reacting a radical producing species with the carbon nanotube salt.

14. The process of claim 13, wherein chemically functionalizing the carbon nanotube salt comprises reacting ozone, dimethylsulfoxide, peroxides, azo compounds, or diazonium compounds with the carbon nanotube salt.

15. The process of claim 1, wherein chemically functionalizing the carbon nanotube salt comprises reacting one or more acyl and/or aroyl peroxides with the carbon nanotube salt.

16. The process of claim 1, wherein chemically functionalizing the carbon nanotube salt comprises reacting R—C(O)O—O(O)C—R′ with the carbon nanotube salt, wherein the R and R′ groups are the same or different and are independently selected from alkyl, alkenyl, alkynyl, alkyl groups containing heteroatoms, alkenyl groups containing heteroatoms, alkynyl groups containing heteroatoms, cycloalkyl, heterocyclyl, cycloalkenyl, aryl, and/or heteroaryl; to provide the chemically functionalized carbon nanotubes, wherein the R and R′ groups are covalently bonded to the carbon nanotubes.

17. The process of claim 16, wherein the R—C(O)O—O(O)C—R′ is selected from benzoyl peroxide, lauroyl peroxide, succinic acid acylperoxide, and/or glutaric acid acylperoxide.

18. The process of claim 15, wherein the R—C(O)O—O(O)C—R′ is selected from acetyl peroxide, n-butyryl peroxide, sec-butyryl peroxide, t-butyryl peroxide, t-pentoyl peroxide, iso-valeryl peroxide, furoyl peroxide, palmitoyl peroxide, decanoyl peroxide, lauroyl peroxide, diisopropyl peroxydicarbonate, butylperoxyisopropyl carbonate, trans-t-butylcyclohexanoyl peroxide, trans-4-cyclohexanecarbonyl peroxide and cyclohexyl peroxydicarbonate, cyclopropanoyl peroxide, cyclobutanoyl peroxide and cyclopentanoyl peroxide, bromobutyryl peroxide, (CCl₃CO₂)₂, (CF₃CO₂)₂, (CCl₃CO₂)₂, (RO(CH₂)ₙCO₂)₂, (RCH═CR′CO₂)₂, RC═CCO₂)₂, (N═C(CH₂)ₙCO₂)₂, where n=1-3, cinnamoyl peroxide, bis(p-methoxybenzoyl)peroxide, p-monomethoxybenzoyl peroxide, bis(o-phenoxybenzoyl)peroxide, acetyl benzoyl peroxide, t-butyl peroxybenzoate, diisopropyl peroxydicarbonate, cyclohexyl peroxydicarbonate, benzoyl phenylacetyl peroxide, butylperoxyisopropyl carbonate, p-nitrobenzoyl peroxide, p-bromobenzoyl, p-chlorobenzoyl peroxide, and bis(2,4-dichlorobenzoyl)peroxide, p-methylbenzoyl peroxide, p-methoxybenzoyl peroxide, o-vinylbenzoyl benzoyl peroxide, and/or exo- and endo-norbornene-5-carbonyl peroxide.

19. The process of claim 1, wherein the degree of functionalization is 1 functional group per 100 nanotube carbons.

20. The process of claim 1, wherein the process is a single-pot process.

21. The process of claim 1, wherein reaction time of functionalizing the carbon nanotube salt is about 30 minutes or less.

22. The process of claim 1, wherein the carbon nanotubes are selected from SWNTs, DWNTs and/or MWNTs.

23. The process of claim 1, wherein the process occurs at a temperature that initiates chemical functionalization.

24. The process of claim 1, wherein the process occurs at about room temperature.

25. The process of claim 1, wherein the carbon nanotube salt is a chemically functionalized carbon nanotube salt.

26. The process of claim 1, wherein the chemically functionalized carbon nanotubes resulting from the process are converted to a chemically functionalized carbon nanotube salt, which is used as the carbon nanotube salt when the process is repeated.

27. The process of claim 13, wherein chemically functionalizing the carbon nanotube salt comprises reacting ozone, dimethylsulfoxide, or peroxides with the carbon nanotube salt.
